(19) United States
(12) Patent Application Publication
(10) Pub. No.: US 2008/0171078 A1
(43) Pub. Date: Jul. 17, 2008

(54) Uniformly sized liposomes

(76) Inventor: Mark Gray, Long Beach, CA (US)

Correspondence Address: CHRISTIE, PARKER & HALE, LLP, PO BOX 7068, PASADENA, CA, 91109-7068, US

(21) Appl. No.: 11/653,621

(22) Filed: Jan. 12, 2007

(51) Int. Cl. A61K 9/127; A61K 31/711

(52) U.S. Cl. 424/450; 514/44; 977/800

(57) ABSTRACT

Compositions suitable for use as site-specific biological vectors are provided and comprise substantially uniformly sized liposomes having very narrow size distributions. In particular, the compositions comprise a collection of liposomes sufficient in quantity to be administered as a pharmaceutical, wherein the liposomes have a mean outer diameter, D, of from 20 nm to 1000 nm, and at least 95% of the liposomes have an outer diameter of from 0.97 D to 1.03 D. Collections of liposomes with even narrower size distributions are also provided.

### CROSS-REFERENCE TO RELATED APPLICATION

This application is related to the U.S. patent application entitled, “Method and Apparatus for Making Uniformly Sized Particles,” filed on an even date herewith, the contents of which are incorporated by reference herein.

### FIELD OF THE INVENTION

The present invention is directed to liposomes having narrow size distributions.

### BACKGROUND OF THE INVENTION

While a great number of therapeutic compounds are discovered every year, the clinical applications of these compounds are often limited by their failure to reach the site of action. A further problem is the toxicity of many drugs at non-target sites. Often, compounds with desirable therapeutic effects have been identified and characterized only to be sidelined by their toxicity profiles. Selective drug targeting would not only reduce systemic toxicity but would also improve drug action by concentrating the therapeutic compound in selected cell or tissue targets. The delivery of drugs to specific target sites is therefore of great interest in clinical science.

Unfortunately, drug delivery technologies have not kept pace with target identification and novel compound synthesis. Delivery problems are especially lacking in the rapidly evolving area of RNA and DNA-based therapeutic intervention.

Increasingly, liposomes are being used to deliver drugs and other agents to target sites in cells. Liposomes are hollow, spherical vesicles comprised of membranes that behave as two-dimensional fluids. In a spherical model, steric stability is heightened at a particular particle diameter for any particular lipid formulation based on the free energies associated with slight deformations of the membrane. Adsorption, spreading, fusion, self-healing, and other mechanical properties of liposomes are recognized as important performance indicators toward their application as delivery vehicles.

In general, liposomes can be formed with outer diameters ranging from 20 to 1000 nm (1 μm), more typically 40-500 nm, with ˜100 nm diameter liposomes being particularly desirable for many biological applications. Liposomes smaller than about 20 nm are physically untenable, while liposomes larger than about 1 μm in diameter tend to be unstable and aggregate over time. Liposome size has a direct effect on payload encapsulation efficiency in the case of an active loading scheme whereby preformed liposomes absorb active ingredients from the surrounding media into their interiors, with smaller sized vesicles being more efficient than larger ones. To a large extent, liposome size determines the sites of action of liposome-cell interaction. Size affects not only how and where the liposomes enter a cell, but also whether they reach a particular cell at all. For some therapeutic applications, efficient tissue targeting requires that the liposomes be able to circulate in the bloodstream for a long period of time until a proper target is encountered.

In vivo, liposomes that are too large as a direct result of the manufacturing process, or that agglomerate into larger units as a result of secondary instabilities in solution, will tend to become entrapped in areas simply based on size. For example, the liver removes larger particles from the bloodstream (larger than 200 nm diameter) because of vasculature sized to act as a physical filter. For this reason, many liposome formulations have been created with liver tissue targeting in mind simply because large particles end up in the liver, and this observation leads to the illusion of a natural affinity of liposomes for liver cells. In fact, oversized liposomes merely become entrapped in the liver because of their size. In any application not targeting the liver, liver localization would have the detrimental effect of removing active material from the intended site of deposition, as well as increasing the likelihood of off-targeting and side effects by misplacing an otherwise therapeutic payload.

In some therapeutic applications, liposomes are administered by intravenous injection, and liposome size—and charge—directly influence the clearance of liposomes from the patient's bloodstream. Generally, the longest half-lives are obtained when liposomes are small in diameter (<0.05 μm). It has also been found that “liquid” vesicles are more rapidly removed from blood circulation than “rigid” ones. The behavior of liposome preparations given by alternative parental routes, such as intraperitoneal, subcutaneous or intramuscular route is also influenced by the distribution of liposome size.

In many therapeutic applications, and particularly in systemic delivery and tissue and cell targeting, liposome size is a critical parameter of therapeutic effectiveness. In order for liposomes to function efficiently as vectors for a given biological application, they need to be as monodisperse as possible, i.e., have as narrow a size distribution as possible. In general liposomes are measured in terms of their (outer) diameters, with little discussion in the literature of internal volume. The literature suggests that a collection of liposomes is considered uniformly sized if the liposomes' outer diameters are polydisperse by only ±10%, i.e., 90-110 nm outer diameters for a collection of liposomes having a mean diameter of 100 nm. The fact that this is considered “good” is shocking, as a difference of 10% in diameter corresponds to roughly a 92% difference in internal volume (if, e.g., one assumes an 8 nm thick lipid layer).

Obtaining the ideal liposome size is therefore a matter of determining the proper chemistry for a given biological application and sizing the particles at exactly those dimensions—a tall order for existing technologies.

Clearly, liposome size distribution is a critical parameter with respect to the pharmacological and pharmacodynamic behavior of biologically active substances that are site-specific targeted in vivo. Although various methods of making small unilamellar vesicles (SUVs) are available, from a process perspective, the formation of stable SUVs with a narrow and predictable size distribution remains a challenge. Commercial liposome sizing systems typically operate by making a number of passes through various size reduction methodologies that use shear force and/or ultrasonic energy dispersion to reduce the size of the liposomes to an approximated average. The most common means of resizing is by passing the liposomes a number of times through a membrane. The production of liposomes with very true homodispersity (i.e., substantially monodisperse), has not been reported, and there is no protocol available in the literature for the production of such particles, let alone a protocol for achieving narrow size distributions under the demanding conditions and in the large volumes required for pharmaceutical production.

An unexpected benefit of the regular sizing of liposomes is the ability to control charge density. Charge density is determined by both the internal payload and external lipid envelope. The lipids comprising the envelope are chosen according to their charge, and the ratio of the constituent lipids is determined according to the charge desired. Determining and quantizing the desired overall charge of the loaded particle is particularly important for delivery of highly charged payload such as DNA. Since DNA payloads are often large, and a single copy of the DNA is loaded per liposome, the negative charge is best neutralized by an envelope of a specific size in order to achieve a desired charge balance. Slight variations in charged liposome size distribution could therefore profoundly affect biodistribution. Considering this fact, and not anticipating that liposomes could be made to have a very uniform size/charge, one author wrote that this factor will serve to “preclude or at least limit the in vivo use of many potentially effective lipid-based DNA delivery vectors.”

The limitations of current technology have a detrimental impact on clinical research and commercial utilization of liposome treatments. When polydisperse liposome formulations are used, valuable markers, isotopes, drugs, and other reagents and payloads are wasted, as they do not reach their intended target and are effectively lost. This retards the development of new therapies (in terms of wasted opportunities and increased time in the lab), and increases the cost of commercial applications (more liposomes are required, as much of the liposomes are the wrong size to be effective).

Accordingly, there is a very strong need in the pharmaceutical, biotechnology, and cosmetic industries for substantially homogenous liposome formulations, particularly unilamellar liposomes that exhibit diameters in the 100 to 200 nm range, and an efficient, robust system for reproducibly generating uniformly sized liposomes. In addition, with current liposome and particle manufacturing techniques, it is exceedingly difficult, if not impossible, to know exactly—or even approximately—how many particles are in a given container of any size. This is because available manufacturing processes are batch processes, and only after the batch is created can a person find out what the yield was, and this is accomplished by running a sample through a particle size analyzer (PSA), or by doing some electron microscopy. Both of these methods are expensive, error-prone, and generally unreliable. A digital manufacturing process would be a significant improvement over the art, as it would enable liposomes and other small particles to be produced with great accuracy and precision.

### SUMMARY OF THE INVENTION

The present invention addresses the need for uniformly sized liposomes and improved processes and apparatus for manufacturing them. According to one aspect of the invention, a composition for delivering a payload to a patient comprises a collection of liposomes sufficient in quantity to be administered as a pharmaceutical, wherein the liposomes have a mean outer diameter, D, of from 20 nm to 1000 nm, and at least 95% of the liposomes have an outer diameter of from 0.97 D to 1.03 D. In one embodiment, the collection of liposomes has a normal distribution of diameters, with a mean outer diameter, D, of from 25 nm to 1000 nm, and a standard deviation ≦0.015 D.

In a second aspect of the invention, an apparatus for making substantially uniformly sized liposomes, and other small particles is provided, and comprises a liquid inlet channel; a liquid outlet channel; a plurality of transverse liquid channels extending from the liquid inlet channel to the liquid outlet channel; a plurality of nozzles in liquid flow communication with the plurality of transverse liquid channels; a plurality of nozzle actuators coupled to the plurality of nozzles; and (optionally) a first collection reservoir coupled to the liquid outlet channel. Preferably, an evaporator, such as a membrane pervaporation unit, is coupled to the collection reservoir, directly or indirectly.

In one embodiment of the invention, substantially uniformly sized droplets are generated using nozzles, actuators, software, and electronics associated with “drop on demand” inkjet printers. By controlling the electric impulses to the actuator(s), very precisely sized volumes of fluid are generated and then ejected as droplets into a laminar flow of a substantially immiscible, or at least no more than sparingly soluble, liquid. The first liquid is then carefully removed to yield substantially uniformly sized liposomes. Thus, in this embodiment, liposomes having a narrow size distribution are made by ejecting well-defined droplets of solvent—containing lipids capable of self assembling into liposomes dissolved, dispersed or suspended therein—through the nozzles of the apparatus into a laminar flow of water or other aqueous medium in the transverse liquid channels; collecting the resulting droplets; and then carefully removing the solvent to facilitate self-assembly of the lipids into liposomes. Advantageously, the liposomes' narrow size distribution is correlated to the initial concentration of lipids-in-solvent and the size of the droplets ejected from the nozzles.

In a third aspect of the invention, a method of making substantially uniformly sized liposomes, and other small particles, is provided, and comprises (a) forming droplets of a first liquid in a laminar flow of a second liquid, each droplet having a volume of from 0.97V to 1.03V, where V is the mean droplet volume and 1 fL≦V≦50 nL, and wherein the first and second liquids are, at most, sparingly soluble (more preferably, substantially immiscible) in one another, and the first liquid contains a solute dissolved, dispersed, or suspended therein; and (b) removing the first liquid to form a plurality of substantially uniformly sized particles. For liposomes, the method comprises forming droplets of a first liquid containing one or more lipids dissolved, suspended, or dispersed therein by ejecting the first liquid into an aqueous laminar flow, wherein the first liquid is no more than sparingly soluble (preferably, substantially immiscible) in water, and wherein each droplet has a volume of from 0.97V to 1.03V, where V is the mean droplet volume and 1 fL≦V≦50 nL; and allowing the lipids to self-assemble into substantially uniformly sized liposomes by removing the first liquid. In one embodiment, the substantially uniformly sized liposomes have a mean outer diameter of from 20 nm to 1 μm.

### BRIEF DESCRIPTION OF THE DRAWINGS

Various aspects, embodiments, and advantages of the invention will become better understood when reference is made to the accompanying drawings, wherein:

FIG. 1 is a schematic diagram of a nozzle ejecting a droplet of liquid into a laminar flow of a second liquid, according to one embodiment of the invention;

FIG. 2 is a schematic, partially exploded view of one embodiment of an apparatus for making substantially uniformly sized particles in accordance with the invention;

FIG. 3 is a plan view of a microfluidics channel plate, a component of one embodiment of the invention;

FIG. 4 is a close-up view of a portion of the plate of FIG. 3 denoted by circle A;

FIG. 5 is a more highly magnified close-up of a portion of the plate shown in FIG. 4 taken along line B-B;

FIG. 6 is an exaggerated schematic view of a nozzle ejecting droplets into a microfluidics channel according to one embodiment of the invention;

FIG. 7 is a sectional view of a portion of FIG. 6, taken along line C-C;

FIG. 8 is a schematic diagram of one embodiment of a pervaporation unit used in the practice of the invention;

FIG. 9 is a schematic diagram of a fluid flow plate according to one embodiment of the invention; and

FIG. 10 is a schematic diagram illustrating other components (reservoirs, pumps, logic controls, etc.) of one embodiment of an apparatus according to the present invention.

### DETAILED DESCRIPTION

In a first aspect of the invention, a composition for delivering a payload to a patient comprises a collection of liposomes sufficient in quantity to be administered as a pharmaceutical, wherein the liposomes have a mean outer diameter, D, of from 20 nm to 1000 nm, and at least 95% of the liposomes have an outer diameter of from 0.97 D to 1.03 D, more preferably from 0.98 D to 1.02 D, most preferably from 0.99 D to 1.01 D. In some embodiments, even tighter size distributions are provided, e.g., at least 96%, at least 97%, at least 98%, or at least 99% of the liposomes have an outer diameter of from 0.97 D to 1.03 D, more preferably from 0.98 D to 1.02 D, most preferably from 0.99 D to 1.0 D—an extraordinarily narrow size distribution for small particles. Such a collection of liposomes is well suited for use as a drug delivery device, as its narrow size distribution should ensure that the liposomes—and any payload carried therein—will be delivered to the desired site(s) within a patient's body.

Unless otherwise noted, “payload” refers to a substance that may be carried by, in, on, or with a liposome. “Patient” is synonymous with “subject,” and refers to a human or non-human, mammalian or non-mammalian animal, and includes in-patient, out-patient, and self-administering individuals. “Pharmaceutical” is used in its broadest sense and refers to a therapeutic, prophylactic, diagnostic, or similar agent or agents, including substances that treat, prevent, or diagnose disease or physical condition, or that function as labels, markers, probes, and the like. A payload can be “administered” to a patient orally, by injection, by inhalation, transdermally, or by any other medically acceptable means for delivering a pharmaceutical. “Sufficient in quantity to be administered as a pharmaceutical” means that the collection of liposomes is large enough to be manipulated and delivered to a patient.

The mean outer diameter of the collection of liposomes is brought as close as desired to a particular value (e.g., 100 nm, 200 nm, etc.), which can be selected so that the liposomes are correctly sized to deliver a payload to a desired cellular site of action. For example, in one embodiment, D=100 nm. More generally, the collection of liposomes has a mean outer diameter, D, of from 20 nm to 1000 nm, from 25 to 500 nm, from 50 to 200 nm, from 75 to 125 nm, from 90 to 110 nm, or (e.g.) from 95 to 105 nm, with a narrow size distribution (optionally Gaussian) about the mean diameter, D; i.e., at least 95%, at least 96%, at least 97%, at least 98%, or at least 99% of the liposomes have outer diameters whose values deviate from D by no more than 3%, 2%, or, most preferably, 1%.

The outer diameters of individual liposomes, as well as the mean outer diameter of a collection of liposomes, can be determined using a suitable measurement technique, for example, photon correlation spectroscopy, freeze fracture and electron microscopy, or by using the “Coulter principle,” whereby voltage potential fluctuation in a small orifice determines particle size. Alternatively, other techniques for determining particle size and size distributions, presently known or discovered in the future, are used. In general, photon correlation spectroscopy is preferred over electron microscopy because it is significantly faster. Advantageously, however, actual measurement is not required, as the method of manufacture is designed to ensure that the liposomes have the desired mean diameter and narrow size distribution, as described below.

It is contemplated that all manner of liposomes can be prepared with a desirably narrow size distribution as described herein, regardless of the lipid(s) and/or other chemical species that comprise the liposomes. The liposomes can be monolayer vesicles, or bilayer vesicles (formed, e.g., of amphipathic, aka amphiphilic, lipids), and can be multilamellar or, more preferably, unilamellar. Phospholipids, such as phosphatidylethanol amines, are a type of amphipathic lipid capable of self-assembling into liposomes in water. A non-limiting list of lipids capable of self-assembling into liposomes is found in U.S. Pat. No. 7,083,572, col. 20, lines 23-59, which is hereby incorporated by reference herein.

The collection of liposomes can be prepared with or without a payload, including payloads that function as biological labels, probes, or markers. Non-limiting examples of payloads include amino acids, proteins, enzymes, natural and synthetic nucleic acids (e.g., DNA, RNA, siRNA, plasmids, etc.), dyes, contrast agents, radiolabeled compounds, fluorescent compounds, medicaments, organic compounds, inorganic compounds (e.g., gold and/or other metallic particles; semiconducting particles, e.g., nanodots), and mixtures of these and/or other substances. The payload can have any desired chemical form, including atomic, molecular, and ionic. In one embodiment, the collection of liposomes is carried in a liquid medium, which typically is water or some other aqueous medium. For example, the aqueous medium can further comprise a pH buffer. Non-limiting examples of buffers include saline, ammonium sulfate, HEPES (4-(2-hydroxyethyl)-1-piperazineethanesulfonic acid), TRIS (tris(hydroxymethyl)aminomethane), and mixtures thereof. The liquid medium itself can also carry or include a payload, which can be the same as or different from the payload carried by the liposomes, if any.

Where a payload is included, it can be carried by or coupled to the liposomes in a number of ways familiar to persons skilled in the art. For example, the payload can be carried within the hollow interior of the liposomes (which, in most cases, also hold water). The payload can be coupled to or carried on the inner and/or outer walls of the liposomes. The payload can be enmeshed within the mono- or bi-layers that form the liposomes. In some embodiments, the payload extends from the interior to the exterior of the liposomes.

As indicated above, in view of the tight size distribution, the collection of liposomes can be characterized by a mean outer diameter and a standard deviation there from. Thus, in one embodiment of the invention, a composition for delivering a payload to a patient comprises a collection of liposomes sufficient in quantity to be administered as a pharmaceutical (for example, 10⁷ or more liposomes), wherein the liposomes have a mean outer diameter, D, of from 25 nm to 1000 nm, with a standard deviation less than or equal to 0.015 D. In some cases, the collection of liposomes will have a spread of outer diameters that can be characterized by a so-called normal distribution, with a probability function, P(x), where x is distance (outer diameter) in nanometers. Systemic errors in the liposomes production process (and/or the manufacturing process(es) used to make various components of the apparatus used to make the collection of liposomes), as well as other factors, may, in some cases, result in the liposomes having a size distribution other than that characterized by the classic Gaussian probability function.

The invention also provides a method and apparatus for making a collection of liposomes—as well as other small particles—having a desired mean outer diameter and a very narrow size distribution. In one embodiment, the method comprises (a) forming droplets of a first liquid in a laminar flow of a second liquid, each droplet having a volume of from 0.97V to 1.03V (preferably 0.98V to 1.02V, more preferably 0.99V to 1.01V) where V is the mean droplet volume, 1 fL≦V≦50 nL, and wherein the first and second liquids are, at most, sparingly soluble in one another, and the first liquid contains a solute dissolved, dispersed or suspended therein; and (b) removing the first liquid to form a plurality of substantially uniformly sized particles. In one embodiment, the liposomes thus formed have a mean outer diameter, D, of from 20 nm to 1000 nm, and at least 95% of the liposomes have an outer diameter of from 0.97 D to 1.03 D, more preferably from 0.98 D to 1.02 D, most preferably from 0.99 D to 1.01 D. In some embodiments, even tighter size distributions are provided, e.g., at least 96%, at least 97%, at least 98%, or at least 99% of the liposomes have an outer diameter of from 0.97 D to 1.03 D, more preferably from 0.98 D to 1.02 D, most preferably from 0.99 D to 1.01 D. In one embodiment, D=100 nm. More generally, the collection of liposomes has a mean outer diameter, D, of from 20 nm to 1000 nm, from 25 to 500 nm, from 50 to 200 nm, from 75 to 125 nm, from 90 to 110 nm, or (e.g.) from 95 to 105 nm. The collection of liposomes has a narrow size distribution (optionally Gaussian) about the mean diameter, D; i.e., at least 95%, at least 96%, at least 97%, at least 98%, or at least 99% of the liposomes have outer diameters whose values deviate from D by no more than 3%, 2%, or, most preferably, 1%.

In the case where the uniformly sized particles are liposomes, the method comprises forming droplets of a first liquid containing one or more lipids dissolved, suspended, or dispersed therein by ejecting the first liquid into a laminar flow of a second liquid, i.e., water or other aqueous medium, wherein the first liquid is no more than sparingly soluble in the second liquid, and wherein each droplet has a volume of from 0.97V to 1.03V (preferably 0.98V to 1.02V, more preferably 0.99V to 1.01V), where V is the mean droplet volume and 1 fL≦V≦50 nL; and allowing the lipids to self-assemble into substantially uniformly sized liposomes by removing the first liquid.

As used herein, the term “sparingly soluble” refers to a solubility of 10% or less, under the conditions of temperature and pressure encountered during droplet formation. In some embodiments, the first and second liquids are substantially immiscible in one another.

To prepare liposomes from lipids capable of self-assembling in water, it is desirable that the first liquid is hydrophobic, such as an organic solvent, and the second liquid is water or some other aqueous medium. Nonlimiting examples of organic solvents include hydrocarbons (e.g., hexane), halogenated hydrocarbons (e.g., dichloromethane, chloroform, trichloroethylene, freon, etc), aromatic compounds (e.g., toluene), and ethers (if sufficiently hydrophobic). Mixtures of organic compounds can be used. The first liquid should be one in which a desired lipid or lipids can be dissolved, dispersed, or suspended. The second liquid may also include a pH buffer, as described above.

The hydrophobicity/hydrophilicty of the first and second liquids also can be reversed from that just described. Thus, in another embodiment of the invention, the first liquid is aqueous and the second liquid is hydrophobic. For example, the second liquid can comprise at least one organic solvent, nonlimiting examples of which are provided above. In still another embodiment, the first liquid or the second liquid comprises mercury, and the opposite liquid (second or first) is selected to be no more than sparingly soluble (preferably, immiscible) therewith.

In the method described, the first liquid contains a solute dissolved, dispersed or suspended therein. Non-limiting examples of solutes include polymers, lipids (especially amphipathic lipids), organic compounds, inorganic compounds, and mixtures thereof. Of particular interest are solutes comprising one or more amphipathic lipids capable of forming bilayer liposomes. One or more additional agents (e.g., detergents, surfactants, antioxidants, etc.) can also be present.

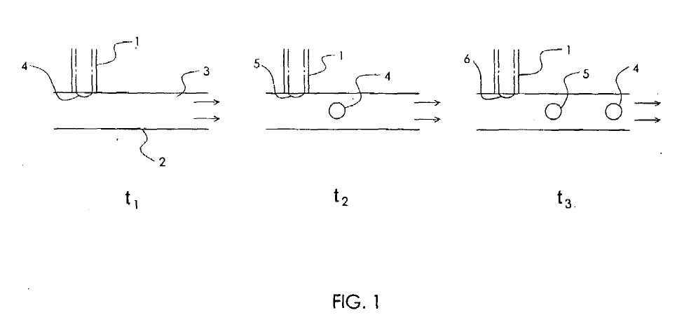

As characterized, the invention contemplates that droplets having a mean volume of from anywhere from 1 fL to 50 nL are formed. In some embodiments, however, the mean droplet volume is confined to a narrower range, namely, from 1 pL to 50 pL. In one embodiment of the invention, droplets of the first liquid are formed by ejecting the first liquid through at least one nozzle directly into a laminar flow of the second liquid. This is schematically illustrated in FIG. 1. A nozzle 1 is in fluid flow communication with a channel 2 through which moves a laminar flow of a second liquid 3. At time t₁, the nozzle eject a first droplet 4 of a first liquid into the channel and is carried downstream by the second liquid. At time t₂, a second droplet 5 of the first liquid is ejected through the nozzle into the channel and also is carried downstream. At time t₃, a third droplet is ejected from the nozzle into the channel and carried downstream. The first, second, and third droplets of the first liquid are substantially immiscible with (or no more than sparingly soluble in) the second liquid, and each droplet is separated in space by a distance determined by the nozzle ejection rate and the rate of laminar flow of the moving liquid in the channel. The flow of liquids in the channel is substantially laminar (non-turbulent), and a sufficient delay is provided between each droplet such that the droplets substantially retain their integrity as they flow through the channel. Consequently, when they are collected downstream and the first liquid is evaporated away (e.g., in a membrane pervaporation unit), the solute that is contained in the droplets becomes concentrated. In the special case where the solute comprises one or more lipids capable of forming liposomes, removal of the first liquid brings the lipids into contact with the aqueous second liquid, and the lipids spontaneously self-assemble and form liposomes, the diameters of which are neatly correlate to the droplet volume and the initial concentration of lipids in the first liquid.

Substantially uniformly sized small particles are obtained by carefully removing the first liquid (or a substantial quantity thereof) from the droplets, so that what is left are discrete particles formed of or from the solute(s), and having a very tight size distribution. The first liquid can be removed in a number of ways. In one embodiment, the first liquid is removed by simple evaporation: the droplets of first liquid carried in the second liquid are drawn off into an open reservoir and allow to off-gas the first liquid. In another embodiment, the first liquid is removed by pervaporation, i.e., membrane pervaporation.

Pervaporation is a separation technique that allows one type of liquid to be separated from an admixture of different liquids. In the case of liposome formation, where the first liquid is an organic (hydrophobic) solvent containing small amounts of solute molecules (lipids), and the second liquid is water, a predominantly aqueous admixture of water and nonpolar liquid (i.e., the droplets of hydrophobic solvent carrying a small concentration of lipids) is brought in contact with a thin, hydrophobic membrane, which is permeable to the solvent, but not to water. The feed (upstream) side of the membrane is more or less at ambient pressure, while the downstream side of the membrane is brought under reduced pressure by, e.g., connecting it to a vacuum pump. The permeate (solvent) is pulled through the membrane and, preferably, captured by a cold trap and, optionally, collected and recycled. As more solvent is removed from the water, the droplets continue to lose more solvent, until substantially all that is left is the retentate: in this case, water and solute particles, or water and particles formed from the solute, e.g., liposomes, which can be drawn off and collected. Pervaporation is particularly suited for liposome formation, where the ratio of solvent to lipids is extremely high and it is desirable to minimize disruption of the droplets as the solvent is stripped away.

Pervaporation membranes can be selected based on the identity and properties of the first and second liquids. Nonlimiting examples of pervaporation membranes include supported and self-supporting (e.g., rigid) materials, for example, ceramic membranes (including coated ceramic hybrids).

Where it is desirable to include one or more payloads in admixture with, or carried by or in, the liposomes, the payload can be introduced in a number of ways. For example, the payload can be carried by the first and/or the second liquid, or introduced into the system after droplet formation, or even after pervaporation.

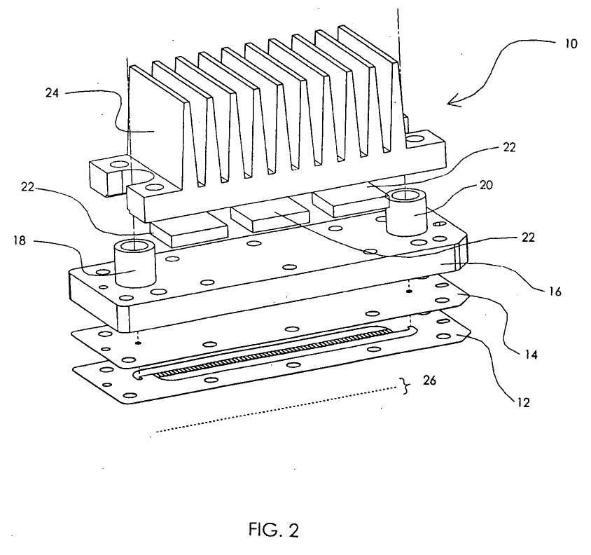

FIGS. 2 through 10 illustrate one embodiment of an apparatus for making substantially uniformly sized liposomes and other small particles according to the invention. Referring to FIG. 2, there is shown an apparatus 10 having a bottom microfluidics channel plate 12, a top microfluidics channel plate 14, an inlet/outlet manifold 16 (having a liquid inlet port 18 at one end and a liquid outlet port 20 at the opposite end), thermoelectric heater/coolers 22, and a radiator 24 (which are thermally coupled to the inlet/outlet manifold and provide a way of supplying and/or removing heat to and/or from the apparatus). A plurality of nozzles 26 are positioned below the bottom microfluidics channel plate and provide a means for ejecting precisely controlled droplets of a first liquid into the apparatus. In the embodiment shown, 64 nozzles are depicted schematically. One or more nozzles can be provided as an inkjet printer head (e.g.) or as stand alone nozzles capable of ejecting discrete droplets. The apparatus also includes one or more nozzle actuators (not shown), which provide a carefully controlled impulse to eject a precisely sized bubble (droplet) of the first liquid through each nozzle. Non-limiting examples of nozzle actuators include piezoelectric actuators and thermal bubble actuators. In one embodiment, each nozzle is driven by a separate actuator. In another embodiment, a single actuator drives two or more nozzles. Inkjet printer heads, nozzles, and actuators, and the associated electronics and software to drive them, are well known in the art.

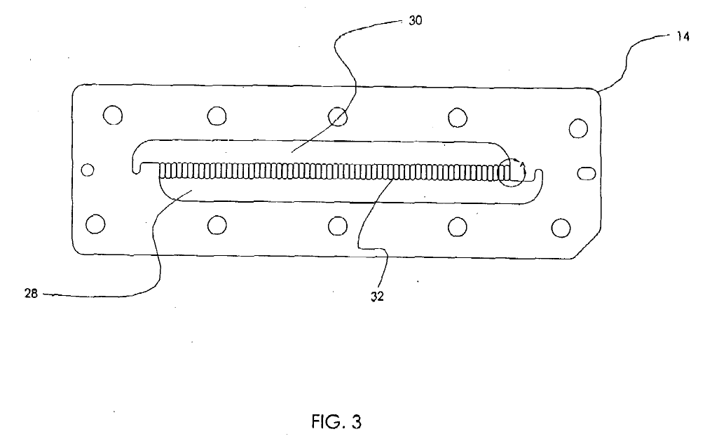

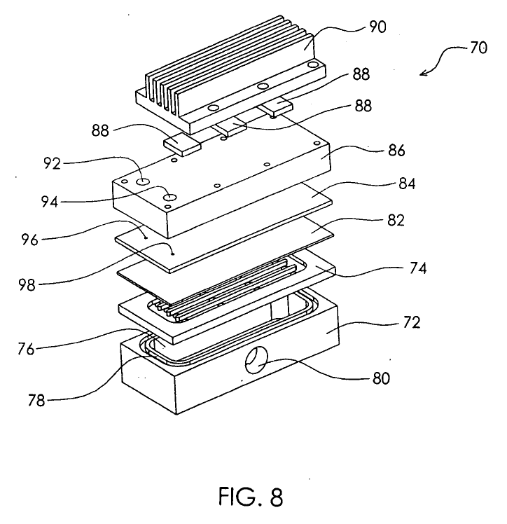

The top and bottom microfluidics channel plates 14, 12 mate in fluid-tight fashion and together form a microfluidics channel at the plates' interface. Referring now to FIG. 3, the top microfluidics channel plate 14 is shown in greater detail. The plate includes an inlet channel 28, an outlet channel 30, and a plurality of transverse liquid channels 32 that extend from the inlet channel to the outlet (exit) channel. In the embodiment shown, 64 such transverse channels are provided. One end of the inlet channel includes an opening that allows liquid to flow into the channel from the inlet port 18 in the inlet/outlet manifold. Similarly, one end of the exit channel has an opening that allows liquid to exit to the outlet port 20 in the inlet/outlet manifold. The outlet port, in turn, can be coupled to a pervaporation unit (FIG. 8), either directly or via a fluid conduit.

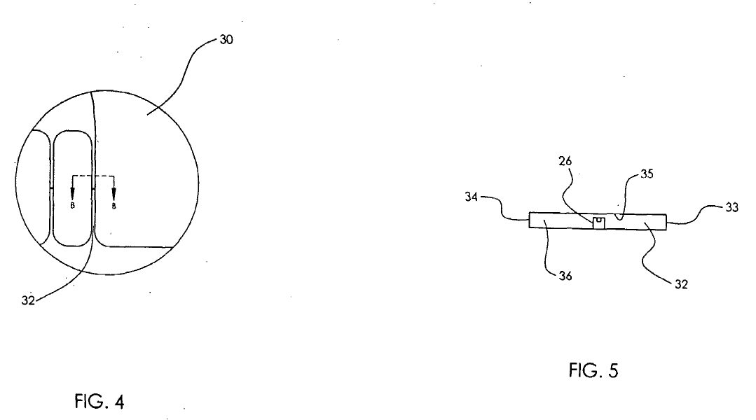

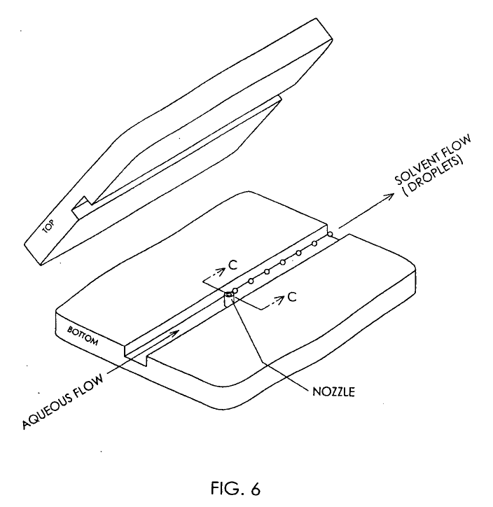

Additional detail of the transverse liquid channels and their relationship with the plurality of nozzles is shown in FIGS. 4 and 5. As indicated, the transverse liquid channels 32 are substantially smaller in cross-section than the inlet and outlet channels. In FIG. 5, a cross-sectional view taken along line B-B, one of the nozzles 26 is shown extending into one of the transverse liquid channels 32. The transverse liquid channel has opposite sidewalls 33, 34, a bottom 35 formed by the bottom microfluidics channel plate, and a top 36 formed by the top microfluidics channel plate. The nozzle extends from the bottom of the transverse liquid channel up into the interior of the channel through an opening (not shown) in the bottom of the bottom microfluidics channel plate.

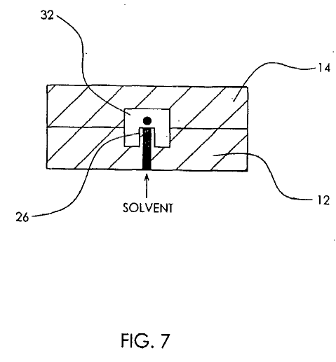

FIGS. 6 and 7 are exaggerated schematic views showing a portion of a top microfluidics channel plate 14 rotated out and away from the bottom microfluidics channel plate 12, revealing one of the plurality of transverse liquid channels 32, with a nozzle 26 protruding up from the bottom of the channel into the interior. This is also shown in FIG. 7, an exaggerated sectional view taken along line C-C.

It will be appreciated that the dimensions of the inlet and outlet channels, the transverse liquid channel, and the nozzles are exceedingly small, with even smaller nozzle diameters (i.e., the inner diameter of the ejection orifice at the tip of each nozzle). Nonlimiting examples include: transverse channels: 1-30 μm (with small dimensions being preferred); inlet and outlet channels: the same size as, or slightly larger than, the transverse channels; nozzle orifices: 0.01-30 μm, preferably 0.01-10 μm. Small transverse channels permit small particles to be obtained and require less buffer in the system, yielding a higher titre of the final composition, i.e., more particles (liposomes) per milliliter. Smaller nozzles permit smaller droplets to be generated, which should yield a greater number of particles in the final composition per unit volume, e.g., more liposomes per mL.

In the embodiment shown in FIGS. 2 through 7, each of the plurality of nozzles has substantially the same nozzle diameter, and each nozzle has a proximal end coupled, directly or indirectly, to one or more liquid reservoirs (not shown), and a distal end that extends into the interior of a corresponding one of the plurality of transverse liquid channels. Alternate embodiments, however, are also within the scope of the invention. For example, the nozzles need not necessarily have the same nozzle diameter. In addition, each nozzle can be flush with the bottom of a corresponding transverse liquid channel, or some nozzles can protrude into, while others are flush with, a corresponding transverse liquid channel, etc. Two or more nozzles can extend into a single transverse channel.

In general, the materials used to construct the microfluidics channel plates are selected to be non-corrosive in the presence of water and organic solvents, and cleaning regimens of soap, steam, and/or chlorides. Nonlimiting examples include NiCo (nickel cobalt alloy) and stainless steel. In one embodiment, the top and bottom plates are held together in a press fit to form a fluid-tight assembly by threaded fasteners (not shown) that span the radiator to the I/O manifold, with the thermoelectric heater/coolers held in compression between them. Optionally, a thermally conductive lubricant can be applied to the upper and/or lower surfaces of the heater/coolers to facilitate heat transfer between the radiator and the I/O manifold.

The thermoelectric heater/coolers allow the temperature of the microfluidics channels and the nozzles to be controlled, which can be desirable for a number of reasons. First, controlling the temperature allows the surface tension at the first liquid/second liquid interface (e.g., the solvent/water interface at the nozzle orifices) to be modulated. If the fluids are cold, the surface tension will be greater. Second, in the microfluidics channels, viscosity is controlled via temperature. Third, in the general mixing of the fluids, it is important to maintain a good separation between the different fluid types. The droplets, if too “hot” might tend to “blur” into the other liquid, due to an increase in solubility.

In some embodiments, where heat-sensitive compounds are present, it is contemplated that the apparatus will be operated above, at, or below room temperature (25° C.), in the range 30 to 200° F. (−1 to 92° C.), with 30 to 80° F. (−1 to 26° C.) being most desirable for most liposome chemistry. It is also contemplated that the pressures of the first and second liquids in the apparatus will be carefully controlled. In one embodiment, each of the liquids has, independently, a pressure of 100 psi or less, e.g., from 10-100 psi; more typically 20-40 psi (excluding the pervaporation unit, which, in one embodiment, is expected to operate at a higher pressure). In another embodiment, either or both liquids have a pressure that exceeds 100 psi. The two liquids can be provided to the droplet generator by a pressurized liquid supply system (described below), which is coupled to the nozzles and the inlet port of the inlet/outlet manifold.

One embodiment of a pervaporation unit is depicted in FIG. 8. The pervaporation unit 70 includes a lower housing 72 and a membrane support plate 74, which together define a vacuum chamber 76. An O-ring (not shown) seated in a channel 78 along the top periphery of the lower housing ensures that a gas-tight seal is maintained between the housing and the membrane support plate. A vacuum port 80 in the lower housing can be connected to a vacuum pump (not shown) and permits gasses to be evacuated from the vacuum chamber to create and maintain a reduced pressure inside the vacuum chamber. In some embodiments, a cold trap (not shown) is located in line between the vacuum port and the vacuum pump, allowing the permeate (e.g., organic solvent) to be captured for disposal or, more preferably, reuse. A selectively permeable membrane 82 is sandwiched between a fluid flow plate 84 and the membrane support plate 74. An inlet/outlet manifold 86 sits atop the fluid flow plate. Thermoelectric heaters/coolers 88 and a radiator 90 allow heat to be supplied to or removed from the pervaporation unit as needed. A fluid inlet port 92 in the inlet/outlet manifold can be coupled to the outlet port of the droplet generator (FIG. 2), while pervaporation products (e.g., liposomes in water, emulsions, solid polymer beads, nanodots, other small particle systems) can be removed from the unit through a fluid outlet port 94. Inlet and outlet ports 96 and 98 are also provided in the fluid flow plate 84, and provide access to the selectively permeable membrane 82. Peripheral components such as a power supply and a microprocessor or other logic controller (not shown) can be coupled to the thermoelectric heaters/coolers and, as with the droplet generator, allow the temperature of fluids in the unit to be closely monitored and controlled. In one embodiment, the flow of fluids through the droplet generator and the pervaporation unit, and the temperature of the fluids, are carefully regulated by a shared microprocessor or other logic controller.

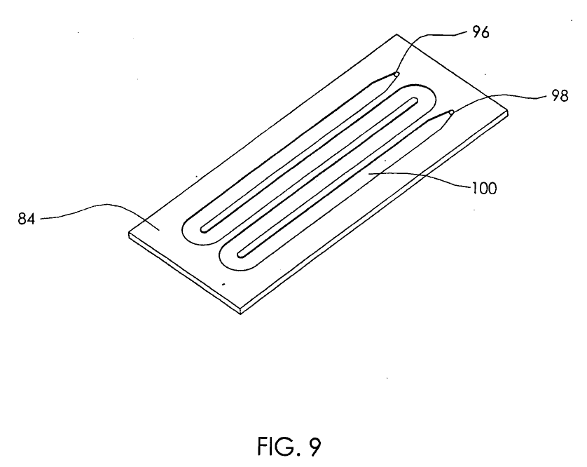

FIG. 9 illustrates the underside of the fluid flow plate 84, which faces the selectively permeable membrane 82. The plate includes openings 96 and 98 and a serpentine channel 100 cut into the lower face, the channel extending from one opening to the other. The serpentine channel provides an extended pathway for a laminar flow of droplets of the first liquid carried by the second liquid, and serves to minimize turbidity while maintaining maximum surface contact between the moving liquids and the selectively permeable membrane. Reduced pressure on the underside of the membrane (facing the enclosed chamber) facilitates the steady, yet controlled, removal of the first liquid along the length of the serpentine channel.

In operation, the pervaporation unit is brought to and maintained at temperature, which may be higher or lower than ambient and within the range of 20 to 170° F. (−7 to 76° C.), as dictated by the physical chemistry of the combination of fluids being separated. For the production of liposomes, in which the lipids must self-assemble, the preferred temperature range is 32 to 80° F. (0 to 26° C.). For the production of other particle types, the range may be much higher because of the inherent stability of the chemistry and more efficient operation of the pervaporation process at higher pressure and temperature differentials. In the case of handling temperature-sensitive molecules or materials, the pervaporation unit is capable of maintaining any temperature with a lower limit defined by the freezing point of the aqueous media.

The pervaporation unit has been designed to remove non-polar solvents from a predominantly aqueous admixture of water and nonpolar solvent(s), by exposing the fluid admixture to a large surface area of a selectively permeable membrane while the other side of the membrane is exposed to a vacuum, or reduced atmospheric pressure. Selective transmission of nonpolar molecules across the membrane is achieved by the material properties of the membrane itself. In this design, a hydrophobic membrane is used to separate solvent from water because nonpolar solvents will be freely absorbed by the membrane to the exclusion of water, which will remain outside of the membrane material. A laminar flow path or paths that minimize the turbidity of the fluid passing within the device while maintaining a maximal surface contact to a large surface area of the membrane material are used to the greatest extent possible. In this way, solvent droplets are able to reduce in volume to a critical point at which the lipid component of the mixture self-assembles into liposomes, with minimal physical disruption caused by shear force in the form of turbidity. To maximize the transmission of the solvent through the hydrophobic membrane, the pervaporation unit has been engineered to withstand pressure differentials of up to 120 PSI across the exposed membrane surface area as well as the ability to acquire and maintain a preset operating temperature within the range of 20 to 170° F. (−7 to 76° C.).

Like the droplet generator, the pervaporation unit is constructed of materials that are non-corrosive in the presence of water and organic solvents, and cleaning regimens of soap, steam, and/or chlorides. In one embodiment, the unit is designed to be serviced and can be disassembled or otherwise opened to allow the selectively permeable membrane to be accessed and replaced in the event it becomes fouled or otherwise rendered unusable.

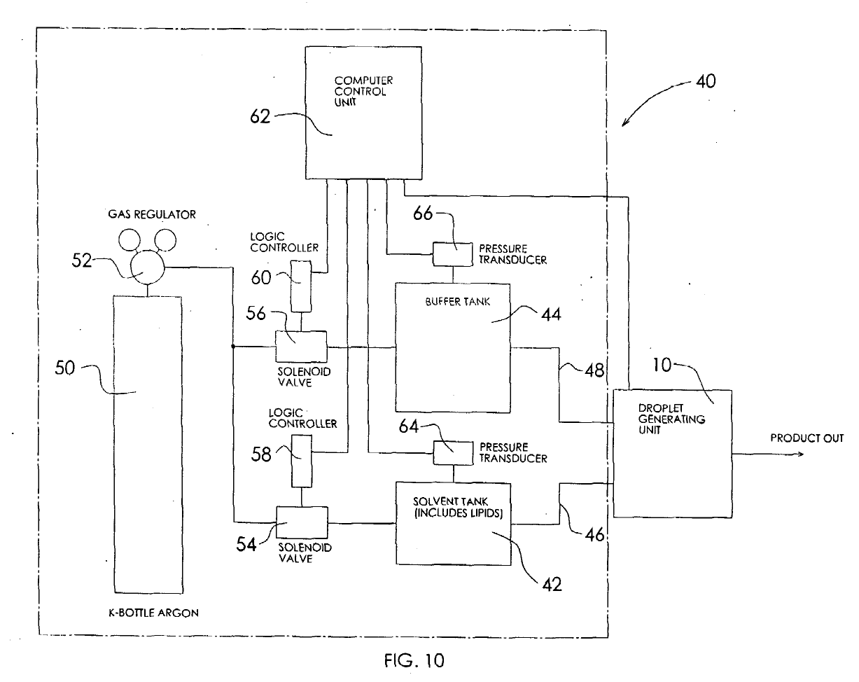

FIG. 10 illustrates one embodiment of a system for delivering pressurized liquids to a droplet generator. The system 40 includes first and second liquid storage tanks 42, 44, which hold, respectively, the first liquid (e.g., an organic solvent from which droplets are formed) and the second liquid (e.g., water or another aqueous medium). For liposome formation, the lipids that will self assemble are also present in the first liquid storage tank, in low concentration. The contents of these tanks are coupled to the droplet generating apparatus 10 by lines 46 and 48 respectively. A gas tank 50, preferably filled with an inert gas, such as argon, neon, helium, etc., is coupled to a gas regulator 52, which in turn is coupled to a pair of solenoid valves 54 and 56. Preferably, each solenoid valve has a small, muffled orifice. The valves allow pressurized gas to be metered into the liquid storage tanks and thereby drive the feed of liquids into the droplet generating unit.

The solenoid valves are coupled to logic controllers 58 and 60 coupled to a computer control unit (e.g., a microprocessor, CPU, computer, etc.) 62, which is coupled to the droplet generating unit and to a pair of pressure transducers 64, 66 associated with the liquid storage tanks 42, 44. (Alternatively, the logic controllers for the solenoid valves are part of the computer control unit.) System commands (i.e., commands for activating/deactivating the nozzle actuator(s) associated with the droplet generator; for controlling the first liquid pressure/flow through the inlet port in the droplet generator; etc.) can be input into the control unit to operate the overall system. A power supply (not shown) is also provided to drive various electrical components of the system.

By controlling the concentration of lipids in the first liquid, the pressures of the first and second liquids, and the size of the droplets generated in the droplet generator, and by carefully removing the first liquid downstream of the droplet generator, a collection of substantially uniformly sized liposomes, having a mean diameter, D, is formed.

Liposome size is a function of the number of molecules comprising the liposome. Likewise, any small particle is sized according to the chemistry and quantity of material comprising the particle, provided, of course, that the material is compacted or otherwise shaped or oriented in such a way that size is a direct function of material volume. Beginning with a specific concentration of solute in a suitable solvent (e.g. lipids in chloroform), each droplet of a specified size will have the same number of lipid molecules distributed in it. Upon removal of the solvent, each liposome or other particle will have substantially the same number of molecules in it, with a variance that is linearly related to the variance in the volume of the droplets. Thus, if the droplets vary in volume by 2-3%, and they are made of the same solution, then particles derived from these droplets will also vary in solute material content and thus, size, by 2-3%.

Controlling particle/liposome size is therefore a function of controlling droplet size and solute concentration. Droplet size is controlled by using nozzles having substantially uniformly sized and shaped ejection orifices, and by adjusting the electrical pulse creating the droplets. The latter is a fine-tuning technique. In one embodiment, droplet volume is corrected (brought toward normality) by up to 10% by adjusting the electrical pulse(s) that drive the nozzle actuator(s). This is similar to the way inkjet printers are adjusted to ensure that each droplet of ink is the same size. In the embodiment shown in FIG. 2, there are 64 nozzles, and each one has a “fingerprint” of sorts. To ensure that each nozzle produces, e.g., a 10 pL droplet, rather than 9 pL droplet, The voltage or current in the pulse wave to the nozzle's actuator is adjusted as necessary. This can be done iteratively. In one embodiment, the apparatus further includes a feedback mechanism in which a computer and particle size detector are used to measure droplet size, compare it to a desired value, and then adjust the electric pulse(s) driving the actuator(s) to correct droplet size as necessary.

One can calculate the size of a particle containing a specified number of molecules if the density or the area in space occupied by a given molecule is known or can be determined. This information can then be used to calculate a specific droplet size, and the corresponding volume of solvent in each droplet will determine the concentration of the starting solvent solution. In theory, particles nearly as large as the droplets themselves can be formed by using very concentrated solutions, at least in the case where the particles are solid, i.e., not liposomes. At the other extreme, very dilute solutions can yield very small particles, e.g., a 100 nm diameter liposome containing just 300,000 lipid molecules is prepared from a very dilute solution of lipids in solvent. Solute concentration can be adjusted directly, by adding additional solute to solvent or by diluting the solution with additional solvent.

In one embodiment of the invention, a relatively concentrated solution of solute in solvent (e.g., lipids in chloroform) is prepared and stored in a first tank. A second tank contains neat solvent (e.g., chloroform). The two tanks are coupled to the droplet generator by one or more conduits and valves which, in turn, are coupled to the system's logic control, so that precise amounts of the solution and solvent can be metered out as needed. The ratio of the solution and neat solvent can be automatically adjusted to produce any desired concentration of solute in solution, from very rich to very dilute. Each of the nozzles can then be selected to eject a particular sized droplet (plus or minus some variance). Alternatively, a variable solvent system is combined with a series of different sized droplet generators, making it possible to achieve any range of particles from big to small, i.e., 5 nm to 100 micrometers for solid particles, and 20 nm-1 micrometer for liposomes.

Advantageously, the process is digital. A computer or other microprocessor issues an electric pulse and a particle is ultimately ejected from the machine. This is a tremendous improvement over the analog processes of open loop hydrodynamic focusing or the condensation reactions that other particle manufacturers use because, in a given run, one will know exactly how many particles were made. Counting very small particles is a serious technical challenge. Ideally, to count the particles in a sample, one might run the entire sample through a particle detector and, each time a particle was detected, an electric pulse would be sent to a counter. The present invention essentially proceeds in the reverse fashion, and thus provides both a particle maker and counter all in one.

### EXAMPLE 1

### Uniformly Sized Phospholipid Liposomes

A droplet generator having 15 micrometer diameter nozzles generates droplets that are 10 pL in diameter and will divide up a liter of chloroform into 1e11 droplets. It will make 1e11 100 nm liposomes. A lipid occupies about 0.4 nm in area in a single layer in a membrane. A 100 nm diameter liposome has an outer (1/2 bilayer) membrane area of 31,400 sq nm. Thus, there are 78,500 lipids in the outer layer. The bilayer membrane is about 5 nm thick, and the inner spherical layer is thus 90 nm in diameter. The inner layer has 25,400 sq nm and thus 63,500 lipid molecules in it. There are therefore 142,000 lipids in this 100 nm liposome.

To make 1e11 100 nm liposomes, 1e11×142,000 lipid molecules are added to one liter of chloroform, with stirring. The molecular weight of a certain phosphatidylethanolamine (C₄₁H₈₃NO₈P) is 749.07), therefore [(1e11)(142,000)(749.07)/(6.022e23)]=0.0000177 grams of lipids are used to make a liter of 2.3e-8 M solution. The solution comprises the “first liquid” in the droplet generator. The second liquid is aqueous, with a small quantity of buffer. The resulting droplets that are formed are passed through a pervaporation unit until substantially all of the chloroform is removed, yielding a collection of substantially uniformly sized (100 nm) liposomes in water.

The invention has been described with reference to various embodiments, figures, and examples, but is not limited thereto. Persons having ordinary skill in the art will appreciate that the invention can be modified in a number of ways without departing from the invention, which is limited only by the appended claims and equivalents thereof.

What is claimed is:

1. A composition for delivering a payload to a patient, comprising a collection of liposomes sufficient in quantity to be administered as a pharmaceutical, wherein the liposomes have a mean outer diameter, D, of from 20 nm to 1000 nm, and at least 95% of the liposomes have an outer diameter of from 0.97 D to 1.03 D.

2. A composition as recited in claim 1, wherein at least 96% of the liposomes have an outer diameter of from 0.97 D to 1.03 D.

3. A composition as recited in claim 1, wherein at least 97% of the liposomes have an outer diameter of from 0.97 D to 1.03 D.

4. A composition as recited in claim 1, wherein at least 98% of the liposomes have an outer diameter of from 0.97 D to 1.03 D.

5. A composition as recited in claim 1, wherein at least 99% of the liposomes have an outer diameter of from 0.97 D to 1.03 D.

6. A composition for delivering a payload to a patient, comprising a collection of liposomes sufficient in quantity to be administered as a pharmaceutical, wherein the liposomes have a mean outer diameter, D, of from 20 nm to 1000 nm, and at least 95% of the liposomes have an outer diameter of from 0.98 D to 1.02 D.

7. A composition as recited in claim 6, wherein at least 96% of the liposomes have an outer diameter of from 0.98 D to 1.02 D.

8. A composition as recited in claim 6, wherein at least 97% of the liposomes have an outer diameter of from 0.98 D to 1.02 D.

9. A composition as recited in claim 6, wherein at least 98% of the liposomes have an outer diameter of from 0.98 D to 1.02 D.

10. A composition as recited in claim 6, wherein at least 99% of the liposomes have an outer diameter of from 0.98 D to 1.02 D.

11. A composition as recited in claim 1, wherein the collection of liposomes comprises 10⁷ or more liposomes.

12. A composition for delivering a payload to a patient, comprising
a collection of liposomes sufficient in quantity to be administered as a pharmaceutical, wherein the liposomes have a mean outer diameter, D, of from 20 nm to 1000 nm, and at least 95% of the liposomes have an outer diameter of from 0.99 D to 1.01 D.

13. A composition as recited in claim 12, wherein at least 96% of the liposomes have an outer diameter of from 0.99 D to 1.01 D.

14. A composition as recited in claim 12, wherein at least 97% of the liposomes have an outer diameter of from 0.99 D to 1.01 D.

15. A composition as recited in claim 12, wherein at least 98% of the liposomes have an outer diameter of from 0.99 D to 1.01 D.

16. A composition as recited in claim 12, wherein at least 99% of the liposomes have an outer diameter of from 0.99 D to 1.01 D.

17. A composition as recited in claim 1, wherein 25 nm≦D≦500 nm.

18. A composition as recited in claim 17, wherein 50 nm≦D≦200 nm.

19. A composition as recited in claim 17, wherein 75 nm≦D≦125 nm.

20. A composition as recited in claim 17, wherein 90 nm≦D≦110 nm.

21. A composition as recited in claim 17, wherein 95 nm≦D≦105 nm.

22. A composition as recited in claim 17, wherein D is 100 nm.

23. A composition as recited in claim 1, wherein at least some of the liposomes carry a payload.

24. A composition as recited in claim 23, wherein the payload is carried within the interior of the liposomes.

25. A composition as recited in claim 23, wherein the payload is carried on and/or in the liposomes' inner and/or outer walls.

26. A composition as recited in claim 23, wherein the payload is selected from the group consisting of amino acids, proteins, enzymes, natural and synthetic nucleic acids, dyes, contrast agents, radiolabeled compounds, fluorescent compounds, medicaments, organic compounds, inorganic compounds, and mixtures thereof.

27. A composition as recited in claim 23, wherein the payload is selected from the group consisting of naturally occurring nucleic acids, synthetic nucleic acids, and mixtures thereof.

28. A composition as recited in claim 23, wherein the payload comprises at least one siRNA.

29. A composition as recited in claim 1, wherein the liposomes are carried in a liquid medium.

30. A composition as recited in claim 29, wherein the liquid is aqueous.

31. A composition as recited in claim 29, further comprising a pH buffer in the medium.

32. A composition as recited in claim 31, wherein the buffer is selected from the group consisting of saline, ammonium sulfate, HEPES, TRIS, and mixtures thereof.

33. A composition as recited in claim 29, further comprising a therapeutic compound carried by the medium.

34. A composition for delivering a payload to a patient, comprising a collection of liposomes sufficient in quantity to be administered as a pharmaceutical, wherein the liposomes have a mean outer diameter, D, of from 20 nm to 1000 nm, with a standard deviation ≦0.015 D.
